(19) United States
(12) Patent Application Publication
(10) Pub. No.: US 2026/0298854 A1
(43) Pub. Date: Oct. 1, 2026

(54) THERMAL CONDUCTIVITY SENSITIVITY COMPENSATION WITH SUPPLY VOLTAGE

(71) Applicant: Infineon Technologies AG, Neubiberg (DE)

(72) Inventors: Dan-Alexandru MOCANU, Bucharest (RO); Silviu-Antonio PIELEANU, Bucharest (RO); Darius-Andrei MANOLESCU, Pitesti (RO)

(21) Appl. No.: 19/568,872

(22) Filed: Mar. 17, 2026

(30) Foreign Application Priority Data
Mar. 25, 2025 (DE) 102025111533.8

(51) Int. Cl. G01N 25/18

(52) U.S. Cl. CPC G01N 25/18

(57) ABSTRACT

A method for operating a thermal conductivity sensor includes the following steps: (i) applying a supply voltage to a measurement element of the thermal conductivity sensor, wherein the supply voltage results in a temperature increase of the measurement element to a characteristic temperature at which the measurement element is sensitive to a thermal conductivity of an analysis gas; (ii) monitoring the supply voltage during the temperature increase; (iii) performing a first measurement by the measurement element during the temperature increase before the measurement element has reached the characteristic temperature, thereby providing a first measurement value; (iv) performing a second measurement by the measurement element at a time when the measurement element has reached the characteristic temperature, thereby providing a second measurement value; and (v) obtaining a compensated measurement value based on the first measurement value, the second measurement value and the monitored supply voltage.

### CROSS-REFERENCE TO RELATED APPLICATION

This application claims priority to Germany Patent Application No. 102025111533.8, filed Mar. 25, 2025, the contents of which are incorporated herein by reference in their entireties.

### TECHNICAL FIELD

The present disclosure relates to methods for operating thermal conductivity sensors. In addition, the present disclosure relates to thermal conductivity sensors configured to perform such methods.

### BACKGROUND

Thermal conductivity sensors may e.g., be used in the automotive sector or a variety of industrial applications. Here, the sensors may provide measurement values specifying a thermal conductivity of an analysis gas. Precise determination of the thermal conductivity of the analysis gas can provide information about a gas concentration, which in turn can be highly safety relevant. For example, the thermal conductivity sensor can be employed to determine and monitor a hydrogen concentration of a vehicle battery system, wherein exceeding a certain threshold of the concentration can constitute an early warning sign for a thermal runaway of the battery. The sensitivity and accuracy of the measurement values may suffer from variations of the supply voltage provided to the sensor. Manufacturers and designers of thermal conductivity sensors are constantly striving to improve their products. In particular, it may be desirable to provide thermal conductivity sensors taking into account variations in the supply voltage in order to provide reliable and accurate measurement results. In addition, it may be desirable to provide suitable methods for operating such thermal conductivity sensors.

### SUMMARY

An aspect of the present disclosure relates to a method for operating a thermal conductivity sensor includes the following steps: (i) applying a supply voltage to a measurement element of the thermal conductivity sensor, wherein the supply voltage results in a temperature increase of the measurement element to a characteristic temperature at which the measurement element is sensitive to a thermal conductivity of an analysis gas; (ii) monitoring the supply voltage during the temperature increase; (iii) performing a first measurement by the measurement element during the temperature increase before the measurement element has reached the characteristic temperature, thereby providing a first measurement value; (iv) performing a second measurement by the measurement element at a time when the measurement element has reached the characteristic temperature, thereby providing a second measurement value; and (v) obtaining a compensated measurement value based on the first measurement value, the second measurement value and the monitored supply voltage.

A further aspect of the present disclosure relates to a thermal conductivity sensor, including: a measurement element; a measurement circuit configured to: perform a first measurement by the measurement element during a temperature increase of the measurement element to a characteristic temperature at which the measurement element is sensitive to a thermal conductivity of an analysis gas and before the measurement element has reached the characteristic temperature, thereby providing a first measurement value, and perform a second measurement by the measurement element at a time when the measurement element has reached the characteristic temperature, thereby providing a second measurement value; a monitoring circuit configured to monitor the supply voltage during the temperature increase; and a compensation circuit configured to obtain a compensated measurement value based on the first measurement value, the second measurement value, and the monitored supply voltage

Those skilled in the art will recognize additional features and advantages upon reading the following detailed description, and upon viewing the accompanying drawings.

### BRIEF DESCRIPTION OF THE DRAWINGS

The present disclosure is illustrated by way of example, and not by way of limitation, in the figures of the accompanying drawings in which like reference numerals refer to similar or identical elements. The elements of the drawings are not necessarily to scale relative to each other. The features of the various illustrated examples can be combined unless they exclude each other.

FIGS. 1A and 1B illustrate a perspective view of a resistor 100 including a hot wire exposed to air and hydrogen, respectively.

FIG. 2 illustrates a circuit diagram of a bridge circuit 200 that may be included in a thermal conductivity resistor in accordance with the disclosure.

FIG. 3 illustrates a schematic of a thermal conductivity sensor 300 in accordance with the disclosure.

FIG. 4 illustrates a timing diagram for a supply voltage applied to the measurement element, a circuit configuration of the measurement element, and a temperature of a bridge circuit of the measurement element during different phases of the sensor operation in accordance with the disclosure.

FIG. 5 illustrates a flowchart of a method for operating a thermal conductivity sensor in accordance with the disclosure.

### DETAILED DESCRIPTION

In this description thermal conductivity sensors (or thermal conductivity gas sensors) in accordance with the disclosure and methods for operating such sensors will be described in detail. Thermal conductivity sensors as described herein may particularly be used as hydrogen sensors for detecting hydrogen and/or hydrogen concentrations. Hydrogen sensors may be used in a variety of applications, such as e.g., in the automotive sector or industrial applications. By way of example, hydrogen sensors may be used for hydrogen exhaust gas detection, exhaust gas monitoring, battery monitoring, hydrogen leakage detection, hydrogen detection in industrial plants, etc.

With a view to achieving climate targets, the automotive industry is promoting and developing the production of hydrogen-powered vehicles. Fuel cell cars can be considered as a breakthrough for electromobility and can heavily contribute to a reduced CO2 emission. Thermal conductivity sensors as described herein improve hydrogen technology and may thus at least partially contribute to achieving climate targets that have been set. The thermal conductivity sensors as described herein provide a simple and efficient way to compensate for inaccurate measurement values caused by a fluctuation in the supply voltage provided to the sensor. Compared to this, production and design of conventional sensors may be more complex and may require a higher number of components, resulting in an increased consumption of resources. The thermal conductivity sensors as described herein save resources and may contribute to energy savings. Improved thermal conductivity sensors in accordance with the disclosure and methods for operating such sensors may contribute to green technology and green power solutions, e.g., climate-friendly solutions providing reduced energy usage.

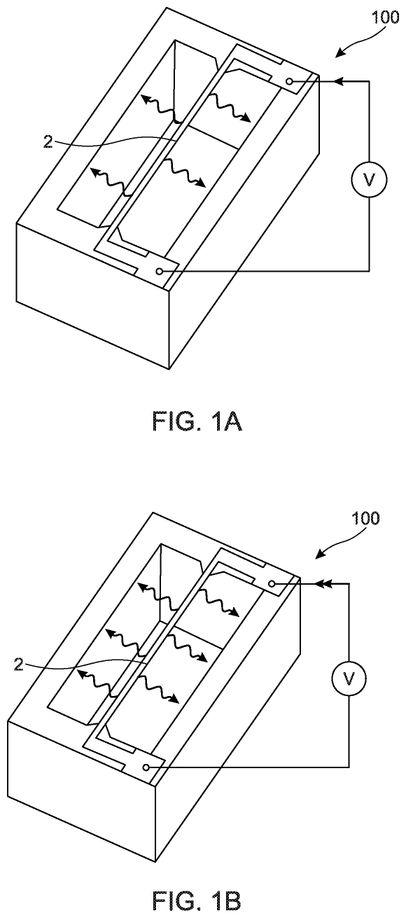

The resistor 100 of FIGS. 1A and 1B may include a hot wire (or heating wire) 2 that may be exposed to a gas. For example, the resistor 100 may be manufactured based on a MEMS (Microelectromechanical systems) technology. When a supply voltage V is applied, the hot wire 2 may heat up to a stable characteristic temperature above ambient temperature. The hot wire 2 may dissipate thermal energy to the surrounding gas as indicated by small arrows pointing away from the hot wire 2. When the stable characteristic temperature is reached, a total heat loss of the resistor 100 (or the hot wire 2) may equal the energy generated by the supply voltage V. Naturally, the heat loss of the resistor 100 may depend on the thermal conductivity of the surrounding gas. The higher the thermal conductivity of the gas, the larger a cooling effect of the surrounding gas and the lower the temperature of the resistor 100 at a constant supply voltage V. In one example, the resistor 100 may be a PTC (Positive Temperature Coefficient) resistor configured to conduct electric currents better at low temperatures than at high temperatures. That is, a resistance value of the resistor 100 may be smaller at low temperatures than at high temperatures. As a result, the electric current through the hot wire 2 may be a measure for the thermal conductivity of the surrounding gas.

FIG. 1A illustrates an operation of the resistor 100 when exposed to air (or ambient air) that may have a nitrogen content of about 78%. Thermal conductivity sensors as described herein may use nitrogen as a reference gas. A resistor exposed to a reference gas may be referred to as reference resistor. Thermal energy dissipated from the hot wire 2 into the surrounding air is indicated by small arrows.

FIG. 1B illustrates a similar operation of the resistor 100 when exposed to an analysis gas (or a gas of interest). A resistor configured to be exposed to an analysis gas may be referred to as sensor resistor. In the example of FIG. 1B, the analysis gas may be or may comprise hydrogen. Note, however, that analysis gases are not restricted to a specific type and may differ in other examples. Compared to FIG. 1A, more thermal energy may be dissipated by the hot wire 2 into the surrounding hydrogen gas as indicated by a greater number of arrows. Accordingly, the electric current through the hot wire 2 in FIG. 1B may be greater than the corresponding electric current in FIG. 1A.

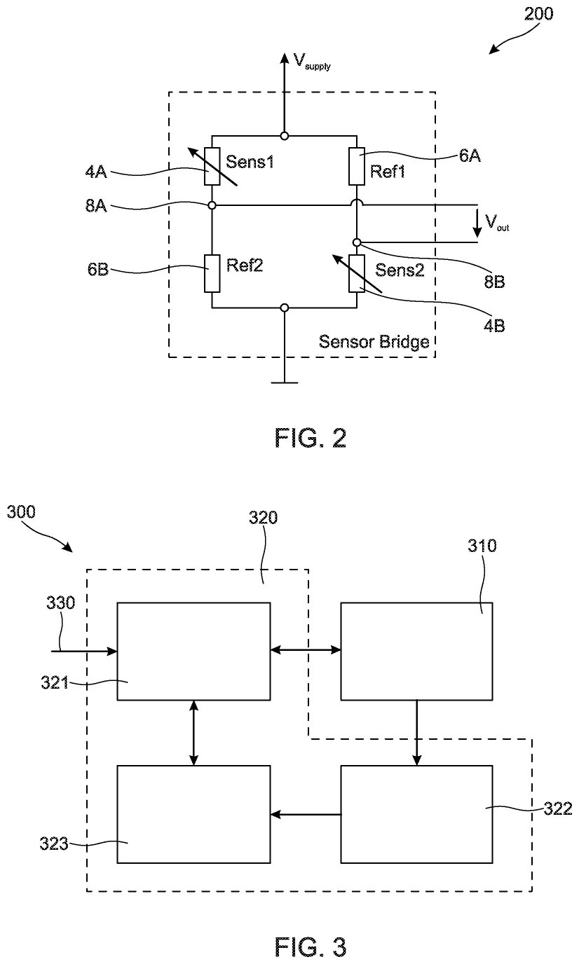

In many applications the electric current through the hot wire 2 may not necessarily be measured directly for analyzing a gas of interest. Instead, one or more sensor resistors exposed to an analysis gas and one or more reference resistors not exposed to the analysis gas may be combined in a half bridge circuit or a bridge circuit. Hereby, a change of thermal conductivity may be turned into a change of a bridge output voltage as discussed in connection with FIG. 2.

FIG. 2 illustrates a circuit diagram of a bridge circuit 200 that may be included in a thermal conductivity resistor in accordance with the disclosure. The bridge circuit 200 may correspond to a Wheatstone bridge circuit including two sensor resistors 4A, 4B and two reference resistors 6A, 6B. The resistors may be interconnected as shown in the circuit diagram. A resistance value of the sensor resistors 4A, 4B may change based on the presence and concentration of an analysis gas. A resistance value of the reference resistors 6A, 6B may substantially remain constant. For example, each of the resistors may be similar to the resistor 100 of FIG. 1.

The bridge circuit 200 may further include a component (not illustrated) for providing a measurement value specifying a thermal conductivity of an analysis gas. In particular, the component may be configured to measure and output a voltage difference Vout between a first node 8A and a second node 8B. The first node 8A may be arranged between the first sensor resistor 4A and the second reference resistor 6B, while the second node 8B may be arranged between the second sensor resistor 4B and the first reference resistor 6A.

During an operation of the bridge circuit 200, a supply voltage Vsupply may be applied as shown in FIG. 2. During a heating phase the bridge circuit 200 may heat up to a characteristic stable temperature at which the bridge circuit 200 may have become (in particular fully) sensitive to a thermal conductivity of the analysis gas. Due to differences in the thermal conductivities of the reference gas and the analysis gas, the bridge circuit 200 may output a non-zero output voltage Vout that may specify the thermal conductivity of the analysis gas. Based on the provided output voltage Vout the analysis gas and/or a concentration of the analysis gas may be determined.

In the following, the bridge circuit 200 may be referred to as measurement element. It is to be noted that the bridge circuit 200 is example and may be replaced by any other half bridge circuit or bridge circuit configured to provide a measurement value specifying the thermal conductivity of an analysis gas. Accordingly, measurement elements as described herein may correspond to or may include a bridge circuit or a half bridge circuit. Thermal conductivity sensors as described herein are not restricted to the example Wheatstone bridge circuit 200 of FIG. 2.

It is to be noted that thermal conductivity sensors as described herein may include further circuit components such as e.g., a switch, a signal amplifier, an analog digital converter, etc. However, such components may not necessarily be regarded as part of a measurement element as exemplarily shown in FIG. 2. An operation of these additional components may not necessarily depend on the present temperature. In contrast to this, an operation of the bridge circuit 200 and the value of the output voltage Vout may be sensitive to the temperature of the bridge circuit.

FIG. 3 illustrates a schematic of a thermal conductivity sensor 300 in accordance with the disclosure. The thermal conductivity sensor 300 may comprise a measurement element 310, which can be a bridge circuit according to FIG. 2. Alternatively, the measurement element can be formed by a thermal conductivity resistor according to FIG. 1A. The thermal conductivity sensor 300 further comprises an electrical control circuit 320 that is coupled to the measurement element 310. The electrical control circuit 320 can be an application-specific integrated circuit, ASIC, which together with the measurement element 310 forms an integrated device. Alternatively, the electrical control circuit 320 is arranged externally to the measurement element 310.

The electrical control circuit 320 comprises a measurement circuit 321 that is coupled to the measurement element 310 and further comprises an interface 330. The interface 330 is coupled to an external device and is configured to receive an external supply voltage VDD,ext for operating the thermal conductivity sensor 300. The interface 330 can further serve as an input and output for receiving a measurement trigger and for outputting an output value, for instance. The measurement circuit 321 is configured to provide a supply voltage VDD,MEMS to the measurement element 310 and to obtain measurement values from the measurement element 310. For example, the measurement circuit 321 is configured to apply the supply voltage VDD,MEMS in order to increase a temperature of the measurement element 310 to a characteristic temperature at which the measurement element 310 is sensitive to a thermal conductivity of an analysis gas. The measurement circuit 321 is further configured to trigger a first measurement by the measurement element 310 during the temperature increase before the measurement element 310 has reached the characteristic temperature, e.g., at the beginning of the temperature increase, and to obtain a first measurement value based on the first measurement. At this first measurement, the contribution due to thermal conductivity of the analysis gas can be negligible compared to offset effects such as a change in temperature, humidity or pressure of the analysis gas, for instance. In other words, the measurement element 310 is substantially insensitive to the thermal conductivity of the analysis gas when performing the first measurement. For example, the measurement element is substantially at ambient temperature when performing the first measurement.

The measurement circuit 321 is further configured to trigger a second measurement by the measurement element 310 at a time when the measurement element 310 has reached the characteristic temperature, and to obtain a second measurement value based on the second measurement. For example, the measurement element 310 comprises a bridge circuit, e.g., a Wheatstone bridge, or a half bridge circuit and the first measurement value and the second measurement value are output voltages of the bridge circuit or the half bridge circuit. For example, the first and second measurement values are obtained by measuring the bridge voltage Vout at the respective points in time during or after the temperature increase, as described with reference to FIG. 2. Moreover, the measurement element 310 can comprise a plurality of resistive elements, and wherein after performing the first measurement and before performing the second measurement, the bridge circuit or the half bridge circuit is switched to a parallel configuration, in which the resistive elements are connected in parallel to each other, and switched to a bridge configuration or a half-bridge configuration during the performing of the first measurement and the second measurement.

The electrical control circuit 320 further comprises a monitoring circuit 322 that is coupled to the measurement element 310 or the measurement circuit 321 and is configured to monitor the supply voltage VDD,MEMS applied to the measurement element 310. For example, the monitoring circuit 322 logs the supply voltage during a measurement time frame. The measurement time frame can be defined as the time, when the supply voltage VDD,MEMS is applied to the measurement element 310 and contains the times, at which the first measurement and the second measurement are performed. More specifically, the monitoring circuit 322 can be configured to determine an amount of thermal energy applied to the measurement element 310 during the measurement time frame. The amount of thermal energy can be determined based on a mean or based on an integration of the supply voltage within the measurement time frame, for instance. In particular, the monitoring circuit 322 is configured to determine a deviation between the supply voltage VDD,MEMS that is actually applied to the measurement element 310 deviation from a nominal supply voltage for the duration of the measurement time frame, e.g., for a time that the supply voltage is applied to the measurement element 310. The monitoring circuit 322 can further or alternatively be configured to determine a compensated characteristic temperature from the monitored supply voltage. For example, the monitoring circuit 322 determines from fluctuations in the applied supply voltage VDD,MEMS an amount of thermal energy fed to the measurement element 310, and further determines an actual characteristic temperature and/or a deviation from a nominal characteristic temperature.

The electrical control circuit 320 further comprises a compensation circuit 323 that is coupled to the monitoring circuit 322 and to the measurement circuit 321 and is configured to receive the first and second measurement values from the measurement circuit 321, and the monitored supply voltage, or a signal derived from the monitored supply voltage, from the monitoring circuit 322. The compensation circuit 323 is further configured to obtain a compensated measurement value based on the first measurement value, the second measurement value, and the monitored supply voltage or the signal derived from the monitored supply voltage. The compensated measurement value can be output by the compensation circuit 323 or provided to the measurement circuit 321 for outputting via the interface 330, for instance. The output in both cases can be the compensated measurement value itself or a signal that is derived from the compensated measurement value, e.g., a value indicating the thermal conductivity of the analysis gas or a gas concentration derived from the compensated measurement value.

Obtaining the compensated measurement value can comprise obtaining a compensated second measurement value based on the second measurement value and the monitored supply voltage, or a signal derived from the monitored supply voltage, and determining the compensated measurement value based on the first measurement value and the compensated second measurement value. For example, the compensation circuit 323 receives from the monitoring circuit 322 the monitored supply voltage, compares the monitored supply voltage to a nominal supply voltage, and compensates the second measurement value for deviations between the monitored supply voltage and the nominal supply voltage. For example, the compensation circuit 323 determines an amount of thermal energy applied to the measurement element 310 from the monitored supply voltage, determines an actual characteristic temperature from the amount of thermal energy, compares the actual characteristic temperature to a nominal characteristic temperature that is achieved if the supply voltage VDD,MEMS is stable and equals a nominal supply voltage during the measurement time frame, and compensates the second measurement value for a deviation between the actual characteristic temperature and the nominal characteristic temperature. Alternatively, the determination of the amount of thermal energy and/or of the actual characteristic temperature can be performed by the monitoring circuit 322 and be provided to the compensation circuit 323. The compensation of the second measurement value can be based on the fact that the thermal conductivity scales cubically with the actual supply voltage applied to the measurement element 310, for instance.

The compensation circuit 323 can be further configured to compensate an offset of the second measurement value or a compensated second measurement value based on the first measurement value. For example, compensating the offset of the (compensated) second measurement value comprises subtracting the first measurement value from the (compensated) second measurement value. The offset can be based on one of reflow stress, long term drift, mechanical stress, pressure and humidity.

A electrical control circuit 320 as described can be employed to compensate a measurement signal for variations or fluctuations in the supply voltage VDD,ext, for instance. Typically, an external supply voltage is provided via an external regulator that stabilizes the external supply voltage VDD,ext, provided to the thermal conductivity sensor 300 with an accuracy within a certain range, e.g., within +−1%, +−5% or +−10%, for instance. As nominal supply voltages are typically in the range between 3V and 10V, e.g., 3.3V or 5.0V, fluctuations by the described amount can have a significant impact on the operation of the sensor and the obtained measurement value. A supply voltage that is greater or less than the nominal voltage can particularly for thermal conductivity sensors lead to an actual characteristic temperature that is different from a nominal characteristic temperature, which in turn can falsify or invalidate the thermal conductivity measurement. Particularly in safety relevant applications in which the thermal conductivity sensor is employed for determining a gas concentration, false readings can have serious consequences, such as a failure to detect a potential thermal runaway of a battery due to inaccurate determination of a hydrogen gas concentration, for instance.

The electrical control circuit 320 as described can for example determine the actual characteristic temperature, e.g., a deviation from a nominal characteristic temperature achievable with a stable nominal input voltage VDD,MEMS, and compensate the measurement values, in particular the second measurement value obtained at the actual characteristic temperature, based on the determined deviation from the nominal characteristic temperature. Hence, the need for a further internal voltage regulator of the control circuit for further stabilization is eliminated. Such an additional regulator would otherwise induce a lower sensitivity due to a further voltage drop, as the sensitivity of the thermal conductivity sensor essentially scales cubically with the achievable temperature difference between the ambient temperature and the characteristic temperature, and hence with the supply voltage VDD,MEMS that can be applied to the measurement element 310. The present disclosure thus provides a solution to fluctuating input voltages while maintaining the fact that the supply voltage VDD,MEMS applied to the measurement element 310 essentially equals the external supply voltage VDD,ext.

The measurement circuit 321, the monitoring circuit 322 and the compensation circuit 323 are illustrated as individual components for illustrative purposes. All components can be comprised by a single control circuit in an actual device.

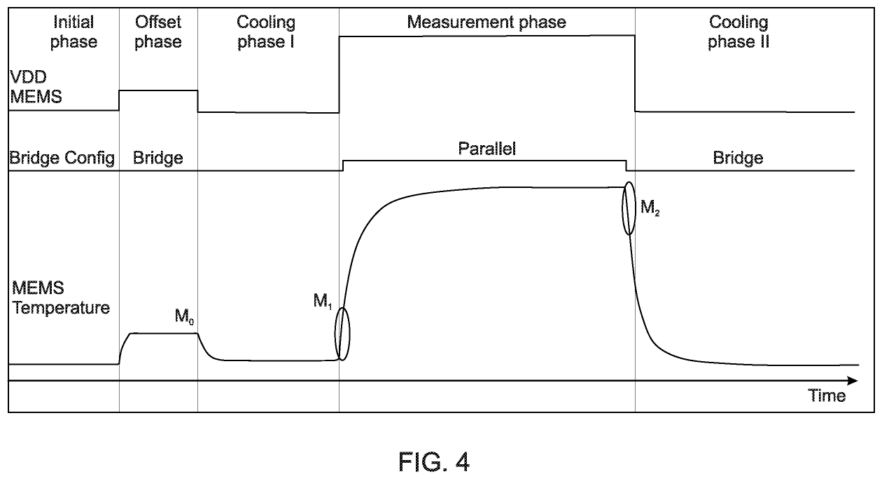

FIG. 4 illustrates a timing diagram for a supply voltage VDD,MEMS applied to the measurement element 310, a circuit configuration of the measurement element 310 and a temperature of a bridge circuit of the measurement element 310 during different phases of the sensor operation in accordance with the disclosure. Therein, the circuit configuration implies an optional feature of the measurement element 310, in which the individual measurement resistors or bridges Sens1, Sens2, Ref1, Ref2 can be switched between the Wheatstone bridge configuration for the actual measurement, and a parallel configuration, wherein all measurement resistors or bridges are connected in parallel to each other such that each respective resistor or bridge experiences the full supply voltage VDD,MEMS for more efficient heating during the measurement phase.

The initial phase can describe an idle sensor, with no voltage VDD,MEMS applied to the measurement element 310, which implies that the MEMS temperature, e.g., the temperature of the measurement element 310, equals an ambient temperature of the sensor. The circuit configuration of the measurement element 310 can be in the bridge configuration as described with reference to FIG. 2. Upon a measurement trigger, the offset phase is initiated, in which a small voltage, e.g., 1.5V, is applied to the measurement element 310, such that the measurement element 310 is heated to a first characteristic temperature that is elevated with respect to the ambient temperature. A measurement value M0 is obtained at this first characteristic temperature during the offset phase. The first characteristic temperature is characterized by the thermal conductivity of the analysis gas being negligible compared to other offset effects that are dominant at this temperature. The circuit configuration of the measurement element 310 can maintain the bridge configuration throughout the offset phase. The offset phase may be characterized by a duration of around 1 ms.

The offset phase is followed by a first cooling phase, during which the supply voltage VDD,MEMS is deactivated such that the temperature of the measurement element 310 decreases towards the ambient temperature. The first cooling phase may be characterized by a duration of around 1 ms.

The first cooling phase is followed by the measurement phase. The beginning of the measurement phase is marked by applying the nominal supply voltage VDD,MEMS to the measurement element 310. The monitoring circuit 322 is configured to monitor the supply voltage during the measurement phase. With the circuit configuration of the measurement element 310 remaining in the bridge configuration, a first measurement of the measurement element 310 is performed and a first measurement value based on this first measurement is obtained. It is noted that performing the first measurement and obtaining the first measurement value can comprise a series of measurements for obtaining multiple measurement values during the onset of the temperature increase for determining the slope of the temperature increase, for instance.

After performing the first measurement and obtaining the first measurement value, the circuit configuration of the measurement element 310 is switched to a parallel configuration for a more efficient temperature increase towards the characteristic temperature. When the characteristic temperature is reached, the circuit configuration of the measurement element 310 is switched to the bridge configuration for performing the second measurement and obtaining the second measurement value. Like for the first measurement, performing the second measurement and obtaining the second measurement value can comprise a series of measurements for obtaining multiple measurement values during the temperature decrease when in the bridge configuration a smaller voltage is applied to the measurement resistors compared to the parallel configuration.

After the second measurement is performed and the second measurement value is obtained, the supply voltage VDD,MEMS is disabled, marking the end of the measurement phase and the beginning of the second cooling phase before the sensor returns to the idle state in the initial phase, for instance. The measurement phase may be characterized by a duration of less than 30 ms, e.g., around 25 ms.

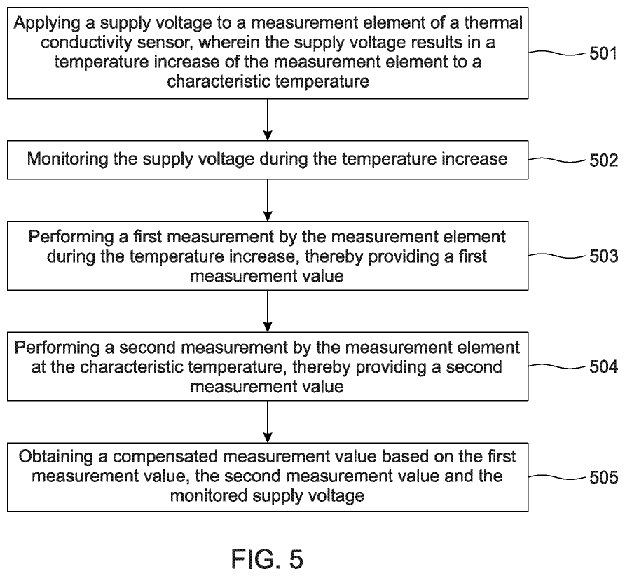

FIG. 5 illustrates a flowchart of a method for operating a thermal conductivity sensor in accordance with the disclosure. The method is described in a general manner in order to qualitatively specify aspects of the disclosure. The method may include further aspects. For example, the method may be extended by any of the aspects described in connection with the timing diagram of FIG. 4.

At 501, a supply voltage may be applied to a measurement element of a thermal conductivity sensor. The supply voltage may result in a temperature increase of the measurement element to a characteristic temperature at which the measurement element is sensitive to a thermal conductivity of an analysis gas. At, 502, the supply voltage applied to the thermal conductivity sensor is monitored using a monitoring circuit. At 503, a first measurement may be performed by the measurement element during the temperature increase before the measurement element has reached the characteristic temperature. In this connection, a first measurement value may be provided. At 504, a second measurement may be performed by the measurement element at a time when the measurement element has reached the characteristic temperature. In this connection, a second measurement value may be provided. At 505, a compensated measurement value may be obtained by compensating the first and the second measurement value based on the monitored supply voltage. The compensation takes into account offset effects, such as changes in relative humidity, pressure or temperature, and also fluctuations and/or variations in the supply voltage applied to the measurement element.

Thermal conductivity sensors in accordance with the disclosure may include components configured to perform the method of FIGS. 4 and 5. In particular, such thermal conductivity sensors may include a measurement element configured to perform steps 503 and 504. In addition, the sensors may include a unit configured to monitor the supply voltage according to step 502, and a unit to obtain the compensated measurement value according to step 505.

In the following, methods for operating thermal conductivity sensors and thermal conductivity sensors in accordance with the disclosure will be explained using examples.

### Aspects

Aspect 1 is a method for operating a thermal conductivity sensor, the method comprising the following steps: (i) applying a supply voltage to a measurement element of the thermal conductivity sensor, wherein the supply voltage results in a temperature increase of the measurement element to a characteristic temperature at which the measurement element is sensitive to a thermal conductivity of an analysis gas; (ii) monitoring the supply voltage during the temperature increase; (iii) performing a first measurement by the measurement element during the temperature increase before the measurement element has reached the characteristic temperature, thereby providing a first measurement value; (iv) performing a second measurement by the measurement element at a time when the measurement element has reached the characteristic temperature, thereby providing a second measurement value; and (v) obtaining a compensated measurement value based on the first measurement value, the second measurement value and the monitored supply voltage.

Aspect 2 is a method of aspect 1, wherein monitoring the supply voltage comprises determining a supply voltage deviation from a nominal supply voltage, and wherein the compensated measurement value is obtained based on the first measurement value, the second measurement value and the supply voltage deviation.

Aspect 3 is a method of aspect 1 or 2, wherein monitoring the supply voltage comprises determining a compensated characteristic temperature, and wherein the compensated measurement value is obtained based on the first measurement value, the second measurement value, and the compensated characteristic temperature.

Aspect 4 is a method of one of the preceding aspects, wherein monitoring the supply voltage comprises determining an amount of thermal energy supplied to the measurement element, and wherein the compensated measurement value is obtained based on the first measurement value, the second measurement value, and the amount of thermal energy.

Aspect 5 is a method of one of the preceding aspects, wherein obtaining the compensated measurement value comprises: obtaining a compensated second measurement value based on the second measurement value and the monitored supply voltage; and determining the compensated measurement value based on the first measurement value and the compensated second measurement value.

Aspect 6 is a method of one of the preceding aspects, wherein obtaining the compensated measurement value comprises compensating an offset of the second measurement value or a compensated second measurement value based on the first measurement value.

Aspect 7 is a method of aspect 6, wherein compensating the offset of the second measurement value comprises subtracting the first measurement value from the second measurement value.

Aspect 8 is a method of aspect 6 or 7, wherein the offset is based on at least one of reflow stress, long term drift, mechanical stress, pressure, humidity.

Aspect 9 is a method of one of the preceding aspects, wherein the measurement element is substantially insensitive to the thermal conductivity of the analysis gas when performing the first measurement.

Aspect 10 is a method of one of the preceding aspects, wherein the measurement element is substantially at ambient temperature when performing the first measurement.

Aspect 11 is a method of one of the preceding aspects, wherein the measurement element comprises a bridge circuit or a half bridge circuit.

Aspect 12 is a method of aspect 11, wherein the first measurement value and the second measurement value are output voltages of the bridge circuit or the half bridge circuit.

Aspect 13 is a method of aspect 11 or 12, wherein the measurement element comprises a plurality of resistive elements, and wherein after performing the first measurement and before performing the second measurement, the bridge circuit or the half bridge circuit is switched to a parallel configuration, in which the resistive elements are connected in parallel to each other, and switched to a bridge configuration or a half-bridge configuration during the performing of the first measurement and the second measurement.

Aspect 14 is a thermal conductivity sensor, comprising: a measurement element; a measurement circuit configured to: perform a first measurement by the measurement element during a temperature increase of the measurement element to a characteristic temperature at which the measurement element is sensitive to a thermal conductivity of an analysis gas and before the measurement element has reached the characteristic temperature, thereby providing a first measurement value, and perform a second measurement by the measurement element at a time when the measurement element has reached the characteristic temperature, thereby providing a second measurement value; a monitoring circuit configured to monitor the supply voltage during the temperature increase; and a compensation circuit configured to obtain a compensated measurement value based on the first measurement value, the second measurement value, and the monitored supply voltage.

Aspect 15 is a thermal conductivity sensor of aspect 14, wherein the measurement element comprises a Wheatstone bridge.

Aspect 16 is a thermal conductivity sensor of aspect 15, wherein the Wheatstone bridge comprises four resistors and each of the resistors comprises a hot wire.

Aspect 17 is a thermal conductivity sensor of aspect 16, further comprising a switching element configured to switch the four resistors between a parallel configuration and a bridge configuration depending on a mode of operation of the thermal conductivity sensor.

Aspect 18 is a thermal conductivity sensor of any of aspects 14 to 17, further comprising an application-specific integrated circuit, ASIC, wherein the ASIC includes at least portions of the measurement circuit, the monitoring circuit and the compensation circuit.

Although specific aspects have been illustrated and described herein, it will be appreciated by those of ordinary skill in the art that a variety of alternate and/or equivalent implementations may be substituted for the specific aspects shown and described without departing from the scope of the present implementation. This application is intended to cover any adaptations or variations of the specific aspects discussed herein. Therefore, it is intended that this implementation be limited only by the claims and the equivalents thereof.

It should be noted that the methods and devices including its preferred implementations as outlined in the present document may be used stand-alone or in combination with the other methods and devices disclosed in this document. In addition, the features outlined in the context of a device are also applicable to a corresponding method, and vice versa. Furthermore, all aspects of the methods and devices outlined in the present document may be arbitrarily combined. In particular, the features of the claims may be combined with one another in an arbitrary manner.

It should be noted that the description and drawings merely illustrate the principles of the proposed methods and systems. Those skilled in the art will be able to implement various arrangements that, although not explicitly described or shown herein, embody the principles of the implementation and are included within its spirit and scope. Furthermore, all examples and implementations outlined in the present document are principally intended expressly to be only for explanatory purposes to help the reader in understanding the principles of the proposed methods and systems. Furthermore, all statements herein providing principles, aspects, and implementations of the implementation, as well as specific examples thereof, are intended to encompass equivalents thereof.

1. A method for operating a thermal conductivity sensor, the method comprising:
applying a supply voltage to a measurement element of the thermal conductivity sensor, wherein the supply voltage results in a temperature increase of the measurement element to a characteristic temperature at which the measurement element is sensitive to a thermal conductivity of an analysis gas;
monitoring the supply voltage during the temperature increase;
performing a first measurement by the measurement element during the temperature increase before the measurement element has reached the characteristic temperature, thereby providing a first measurement value;
performing a second measurement by the measurement element at a time when the measurement element has reached the characteristic temperature, thereby providing a second measurement value; and
obtaining a compensated measurement value based on the first measurement value, the second measurement value, and the monitored supply voltage.

2. The method of claim 1, wherein monitoring the supply voltage comprises determining a supply voltage deviation from a nominal supply voltage, and wherein the compensated measurement value is obtained based on the first measurement value, the second measurement value and the supply voltage deviation.

3. The method of claim 1, wherein monitoring the supply voltage comprises determining a compensated characteristic temperature, and wherein the compensated measurement value is obtained based on the first measurement value, the second measurement value, and the compensated characteristic temperature.

4. The method of claim 1, wherein monitoring the supply voltage comprises determining an amount of thermal energy supplied to the measurement element, and wherein the compensated measurement value is obtained based on the first measurement value, the second measurement value, and the amount of thermal energy.

5. The method of claim 1, wherein obtaining the compensated measurement value comprises:
obtaining a compensated second measurement value based on the second measurement value and the monitored supply voltage; and
determining the compensated measurement value based on the first measurement value and the compensated second measurement value.

6. The method of claim 1, wherein obtaining the compensated measurement value comprises compensating an offset of the second measurement value or a compensated second measurement value based on the first measurement value.

7. The method of claim 6, wherein compensating the offset of the second measurement value comprises subtracting the first measurement value from the second measurement value.

8. The method of claim 6, wherein the offset is based on at least one of reflow stress, long term drift, mechanical stress, pressure, or humidity.

9. The method of claim 1, wherein the measurement element is substantially insensitive to the thermal conductivity of the analysis gas when performing the first measurement.

10. The method of claim 1, wherein the measurement element is substantially at ambient temperature when performing the first measurement.

11. The method of claim 1, wherein the measurement element comprises a bridge circuit or a half bridge circuit.

12. The method of claim 11, wherein the first measurement value and the second measurement value are output voltages of the bridge circuit or the half bridge circuit.

13. The method of claim 11, wherein the measurement element comprises a plurality of resistive elements, and
wherein, after performing the first measurement and before performing the second measurement, the bridge circuit or the half bridge circuit is switched to a parallel configuration, in which the resistive elements are connected in parallel to each other, and switched to a bridge configuration or a half-bridge configuration during the performing of the first measurement and the second measurement.

14. A thermal conductivity sensor, comprising:
a measurement element;
a measurement circuit configured to:
perform a first measurement by the measurement element during a temperature increase of the measurement element to a characteristic temperature at which the measurement element is sensitive to a thermal conductivity of an analysis gas and before the measurement element has reached the characteristic temperature, thereby providing a first measurement value, and
perform a second measurement by the measurement element at a time when the measurement element has reached the characteristic temperature, thereby providing a second measurement value;
a monitoring circuit configured to monitor a supply voltage during the temperature increase; and
a compensation circuit configured to obtain a compensated measurement value based on the first measurement value, the second measurement value, and the monitored supply voltage.

15. The thermal conductivity sensor of claim 14, wherein the measurement element comprises a Wheatstone bridge.

16. The thermal conductivity sensor of claim 15, wherein the Wheatstone bridge comprises four resistors and each of the resistors comprises a hot wire.

17. The thermal conductivity sensor of claim 16, further comprising a switching element configured to switch the four resistors between a parallel configuration and a bridge configuration depending on a mode of operation of the thermal conductivity sensor.

18. The thermal conductivity sensor of claim 14, further comprising an application-specific integrated circuit, ASIC, wherein the ASIC includes at least portions of the measurement circuit, the monitoring circuit and the compensation circuit.
